(19) United States
(12) Patent Application Publication
(10) Pub. No.: US 2022/0257373 A1
(43) Pub. Date: Aug. 18, 2022

(54) ANCHOR FOR PROSTHETIC CARDIAC VALVE DELIVERY DEVICES AND SYSTEMS

(71) Applicant: Shifamed Holdings, LLC, Campbell, CA (US)

(72) Inventors: Alice YANG, Campbell, CA (US); Jonathan OAKDEN, San Jose, CA (US); Jasper Ellington ADAMEK-BOWERS, San Francisco, CA (US); Ali SALAHIEH, Saratoga, CA (US); Savina Brachthauser BALCELLS, Campbell, CA (US); Jordan SKARO, San Jose, CA (US)

(21) Appl. No.: 17/651,040

(22) Filed: Feb. 14, 2022

Related U.S. Application Data
(60) Provisional application No. 63/149,205, filed on Feb. 12, 2021.

(51) Int. Cl. A61F 2/24

(52) U.S. Cl. CPC A61F 2/2418; A61F 2220/0008; A61F 2/2436

(57) ABSTRACT

A device for treating a diseased native valve in a patient is provided, the device including a frame structure and a plurality of leaflets. The device can further include a spiral anchor configured to extend around an outer circumference of the frame structure, the spiral anchor comprising a spiral portion and a proximal portion that extends radially outwards relative to a circumference of the spiral portion. Other embodiments and methods of use are also provided.

### CROSS REFERENCE TO RELATED APPLICATIONS

This application claims the benefit of U.S. Provisional Application No. 63/149,205, filed Feb. 12, 2021, which is incorporated herein by reference in its entirety for all purposes.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Blood flow between heart chambers is regulated by native valves—the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully prolapse, causing the patient to experience regurgitation.

While medications may be used to treat diseased native valves, the defective valve often needs to be repaired or replaced at some point during the patient's lifetime. Existing prosthetic valves and surgical repair and/or replacement procedures may have increased risks, limited lifespans, and/or are highly invasive. Some less invasive transcatheter options are available, but most are not ideal. A major limitation of existing transcatheter mitral valve devices, for example, is that the mitral valve devices are too large in diameter to be delivered transeptally, requiring transapical access instead. Furthermore, existing mitral valve replacement devices are not optimized with respect to strength-weight ratio and often take up too much space within the valve chambers, resulting in obstruction of outflow from the ventricle into the aorta and/or thrombosis.

Thus, a new valve device that overcomes some or all of these deficiencies is desired.

### SUMMARY OF THE DISCLOSURE

In some embodiments, a prosthesis for treating a diseased native valve is provided, the prosthesis comprising a frame structure having a plurality of leaflets therein, and a spiral anchor configured to extend around an outer circumference of the frame structure, the spiral anchor comprising a spiral portion and a proximal portion that extends radially outwards relative to a circumference of the spiral portion.

In some embodiments, the proximal portion crosses the spiral portion to extend beyond the circumference of the spiral portion.

In one embodiment, the proximal portion further extends at a 60°-90° angle relative to a plane of the spiral portion.

In another embodiment, the proximal portion has a length of up to 5 mm. In another embodiment, the proximal portion has a length of up to 10 mm.

In some examples, the proximal portion has a smaller cross-sectional area than that of the spiral portion.

In another embodiment, the proximal portion comprises an abutment at the transition between the proximal portion and the spiral portion.

In some examples, the prosthesis further includes a delivery tether configured to releasably engage with the proximal portion of the spiral anchor.

In one embodiment, the spiral portion is substantially contained within the plane.

A prosthesis for treating a diseased native valve is provided, the prosthesis comprising a frame structure having a plurality of leaflets therein, and a spiral anchor configured to extend around an outer circumference of the frame structure, the spiral anchor comprising a spiral portion and a proximal extension at a proximal end of the spiral portion, the proximal extension extending at a 60°-90° angle relative to a plane of the spiral portion.

In some embodiments, the proximal extension is configured to extend along an outer surface of the frame structure.

In some embodiments, the frame structure comprises a flared proximal portion, the proximal extension terminating distal to the flared proximal portion.

In one embodiment, the proximal extension is configured to position the proximal end to extend at a 60°-90° angle relative to the plane of the spiral portion.

In another example, the proximal extension is less than 10 mm long.

A prosthesis for treating a diseased native valve is provided, the prosthesis comprising a frame structure having a plurality of leaflets therein, and a spiral anchor configured to extend around an outer circumference of the frame structure, the spiral anchor having a proximal portion that is more flexible than a distal portion.

In some embodiments, the proximal portion is thinner or has a smaller cross-sectional area than that of the distal portion.

In some examples, a cross-sectional area of the spiral anchor increases along its length from the proximal portion to the distal portion.

In one embodiment, the increasing cross-sectional area of the spiral portion is gradual along an angular extent of the spiral portion.

In another embodiment, the increasing cross-sectional area of the spiral portion is gradual along a linear extent of the spiral portion.

In some examples, the increasing cross-sectional area of the spiral portion is abrupt along an angular extent of the spiral portion.

In one embodiment, the increasing cross-sectional area of the spiral portion is abrupt along a linear extent of the spiral portion.

A method of delivering a valve prosthesis, comprising the steps of coupling a delivery tether to a proximal portion of a spiral anchor, providing the spiral anchor into a delivery sheath, advancing the delivery sheath to a target tissue location, deploying the spiral anchor from the delivery sheath into a plane to encircle one or more mitral leaflets and chordae at the target location, delivering a frame structure over the delivery tether into place within the spiral anchor, and adjusting an axial position of the spiral anchor relative to the frame structure by the delivery tether while maintaining the planarity of the spiral anchor.

In some embodiments, the axial position of the spiral anchor is adjusted without rotating the spiral anchor relative to the frame structure.

In other embodiments, the axial position of the spiral anchor is adjusted without increasing a radius of curvature of the spiral anchor.

In one example, the axial position of the spiral anchor is adjusted without increasing a radius of curvature of a proximal end portion of the spiral anchor.

A prosthesis for treating a diseased native valve, the prosthesis comprising a frame structure having a plurality of leaflets therein, and a spiral anchor configured to extend around an outer circumference of the frame structure, the spiral anchor comprising a spiral portion that resides in a plane and a proximal valve engagement arm that gradually transitions out of the plane of the spiral portion over approximately 90 degrees to 270 degrees of a circumference of the spiral anchor.

In some embodiments, the proximal valve engagement arm contours closely to the frame structure.

In one embodiment, the proximal valve engagement arm is configured to reduce an axial load on a delivery device of the spiral anchor.

In some examples, a proximal end of the proximal valve engagement arm is positioned in a substantially central point in the spiral anchor to maintain planarity of the spiral anchor as it is moved axially by a delivery device.

### BRIEF DESCRIPTION OF THE DRAWINGS

FIGS. 1A-1B are side and top-down views of an anchor and frame structure.

FIGS. 2A-2H illustrate on method for delivering an anchor and frame structure to a target tissue location.

FIGS. 3A-3C illustrate one embodiment of a spiral anchor including a proximal portion.

FIGS. 4A-4B illustrate another embodiment of a spiral anchor including a proximal portion.

FIGS. 5A-5D illustrate one embodiment of a spiral anchor having a proximal arm extension.

FIGS. 6A-6E illustrate another embodiment of a spiral anchor having a proximal portion.

FIGS. 7A-7B illustrate another embodiment of a spiral anchor having a proximal portion.

### DETAILED DESCRIPTION

Described herein are systems, devices, or methods for treatment or replacement of a diseased native valve of the heart, for example a mitral valve.

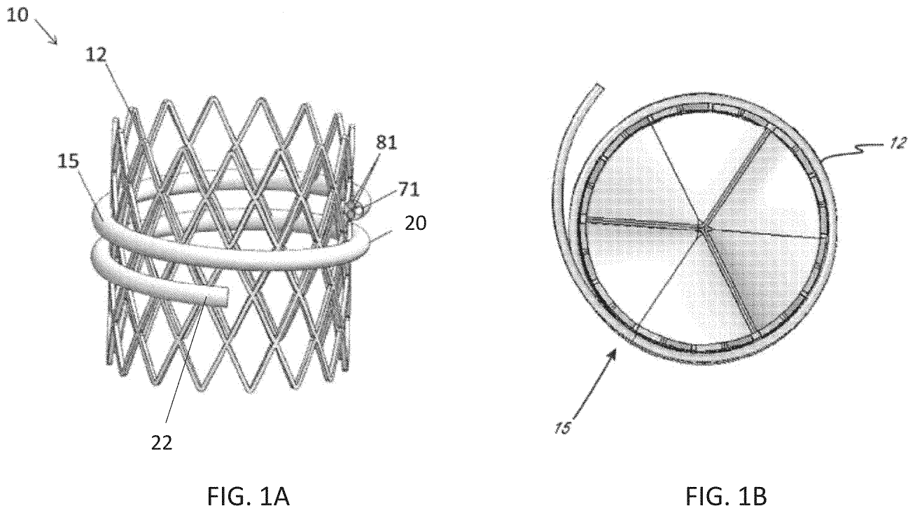

FIGS. 1A-1B show an exemplary valve prosthesis 10 (also referred to herein as “valve device”) for replacement of a valve, such as a mitral valve. The illustrated valve prosthesis 10 comprises a frame structure 12, leaflets 14, and an anchor 15. The anchor 15 includes a wire 20 formed in a spiral shape around the frame structure 12.

The exemplary frame structure 12 is configured like a stent. The frame structure 12 has an expanded state and an unexpanded (e.g., collapsed or compressed) state. The compressed state is sized and dimensioned for percutaneous insertion and the expanded state is sized and dimensioned for implantation in a native valve of a patient, such as a mitral valve.

The anchor 15 can include a spiral member, such as wire 20, having a proximal end 21 and a distal end 22. The anchor 15 can be configured to engage with the frame structure 12 via a compression fit. The wire 20 can be formed of a material having sufficient rigidity to hold a predetermined shape. In an exemplary embodiment, the wire 20 can be formed of a shape memory material (e.g., NiTi). Further, the anchor 15 prior to implantation may comprise a flat spiral shape such that loops of the anchor are generally positioned within the same plane (the plane being perpendicular to a longitudinal axis of a delivery device). Additionally, in some embodiments, the distal end 22 can be rounded and/or atraumatic.

The valve prosthesis 10 can be configured for replacing a mitral valve with the distal end 22 configured for insertion through a commissure.

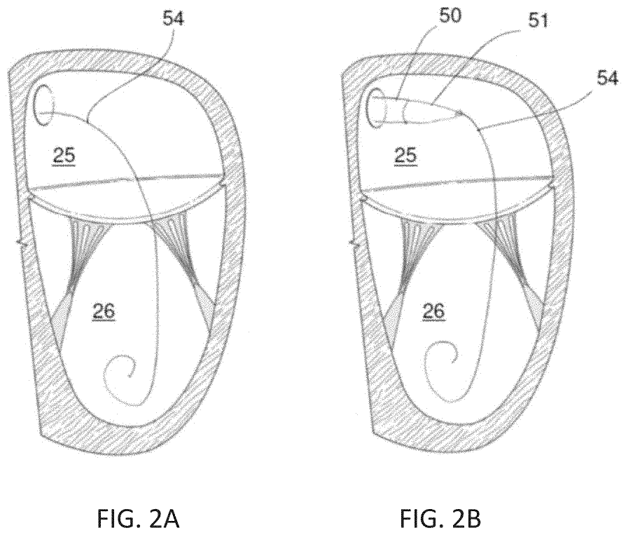

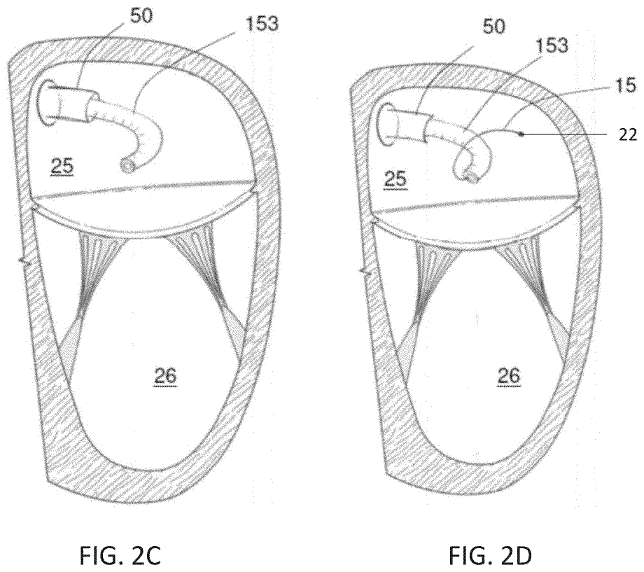

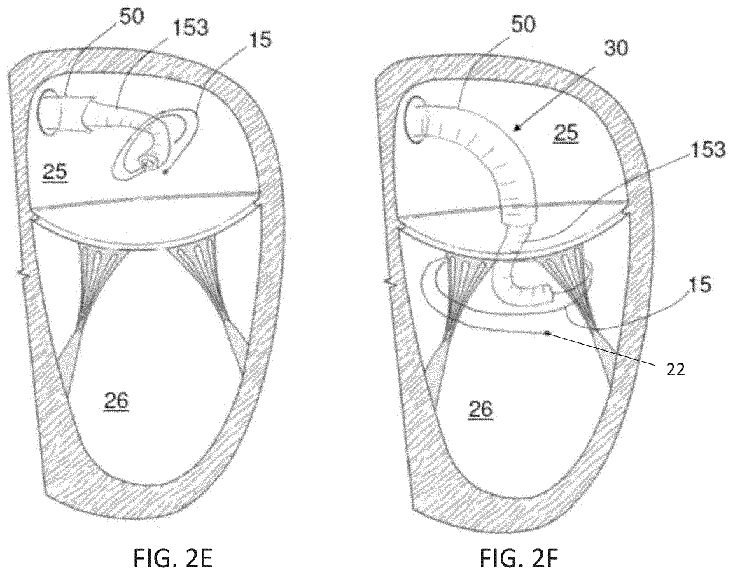

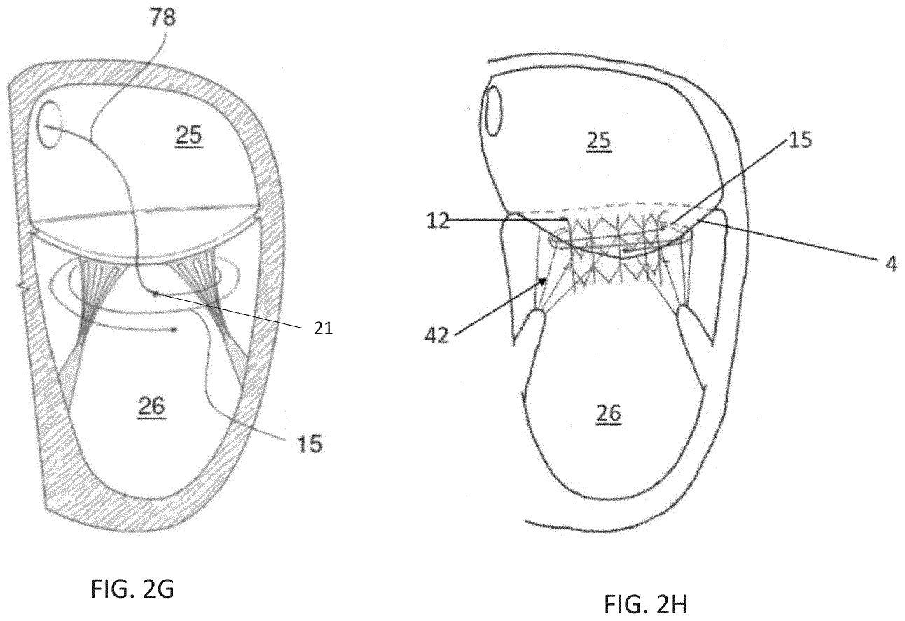

FIGS. 2A-2H show sequential views of an exemplary method of implanting a valve prosthesis 10. At FIG. 2A, a transseptal puncture is made. A guidewire 54 is then routed through the puncture site and left either in the left atrium 25 or across the mitral valve into the left ventricle 26. At FIG. 2B, the outer sheath 50 (optionally with an inner dilator 51) is tracked over the guidewire 54 until the distal end of the outer sheath 50 protrudes into the left atrium 25. The guidewire 54 and inner dilator 51 are then removed from the outer sheath 50. At FIG. 2C, an inner shaft and attached distal anchor guide 153 are inserted through the outer sheath 50 until the distal tip of the anchor guide 153 extends into the left atrium 25. The anchor guide 153 can be positioned and/or oriented as desired by steering the distal end of the sheath 50 and/or rotating the inner shaft and anchor guide 153 relative to the sheath 50. At FIG. 2D, once the anchor guide 153 is in the correct orientation, the anchor 15 can be pushed out through distal tip of the anchor guide 153 (with the distal tip 22 extending out of the guide 153 first). At FIG. 2E, the anchor 15 can fully deploy into the atrium 25. At FIG. 2F, the entire delivery system 30 can be pushed and steered (for example, via steering mechanisms in the outer sheath 50) towards an apex of the ventricle 26, crossing through the mitral valve. In some embodiments, counter-rotation of the anchor 15 may aid in getting the anchor 15 across the mitral valve without tangling. Once the anchor 15 is at the correct depth within the ventricle 26, forward rotation of the anchor 15 (via forward rotation of the inner shaft and guide 153) will allow the anchor 15 to encircle the mitral leaflets and chordae (i.e., with the distal end 22 leading the encircling). At FIG. 2G, the outer sheath 40, inner sheath, and anchor guide 153 are removed, leaving a tether 78 in place (and attached to the proximal end 21 of the anchor 15). At FIG. 2H, the frame structure 12 can then be delivered over the tether 78 and into place within the anchor 15. The tether 78 can then be released from the proximal end 21 of the anchor 15 to leave the prosthesis 10 in place in the mitral valve 4.

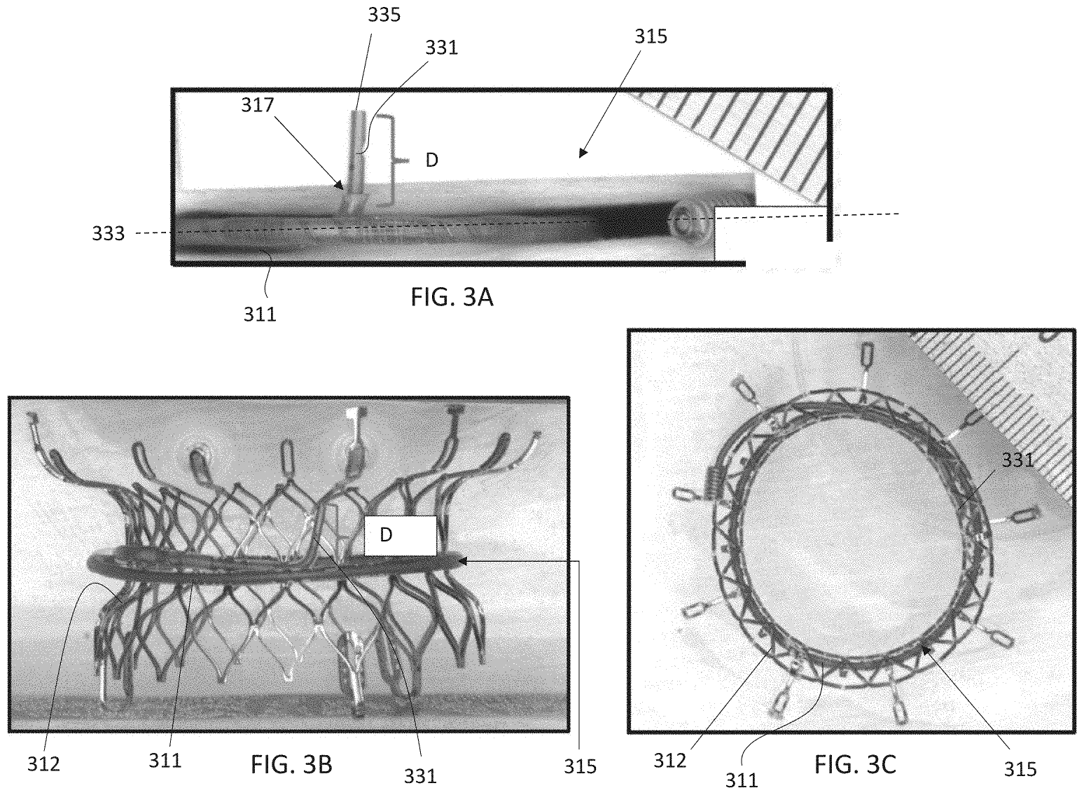

Referring to FIGS. 3A-3C, in some embodiments, the anchor 315 can include a proximal extension 331 configured to extend proximally a distance D from the plane 333 of the anchor spiral when the anchor 315 is implanted (e.g., extend towards the delivery device and/or atrium). In some embodiments, the proximal extension can extend a distance D ranging from 1 mm to 10 mm, or alternatively a distance of approximately 5 mm. Further, the proximal extension 331 can be positioned out of plane relative to the plane 333 of the anchor spiral 311. For example, the proximal extension 331 can extend proximally at an angle of approximately 60°-90° relative to the plane 333 of the spiral 311. The proximal extension 331 can advantageously create a stiff bend or elbow in the anchor 315 that can be used to pull the anchor 315 proximally or push the anchor 315 distally while maintaining the planarity of the spiral 311 relative to the anatomy and/or the frame structure 312. For example, the spiral 311 can maintain its relative planarity (i.e., not tilt or tip) when the proximal extension 331 is pulled proximally by the tether 78 (shown in FIG. 2G) during placement of the anchor 315 in the native valve. Additionally, having a proximal extension 331 that extends out of plane of the spiral 311 can advantageously ensure that the spiral 311 remains encircled (e.g., about the chordae) even when pushing or pulling on the proximal extension 331 (e.g., with the tether 78). Further, in some embodiments, because the proximal extension 331 extends at a steep angle (e.g., 60°-90°) relative to the plane 333 of the spiral 311, the release from the tether 78 can be simple (e.g., because the tether 78 and the proximal extension 331 can be substantially axially aligned).

Referring to the embodiment of FIG. 3A, in some embodiments the proximal extension 331 can have a smaller cross-sectional area (e.g., cross-sectional diameter) than the rest of the anchor 315. In some embodiments, the proximal extension 331 can include an abutment 317 marking the transition between the reduced cross-sectional diameter of the proximal extension and the larger cross-sectional diameter of the anchor. In additional embodiments, the abutment 317 can be used as an engagement point by the tether against the anchor, such as for positioning the anchor in the anatomy or around the frame structure.

As shown in FIG. 3B, the proximal extension 331 can extend alongside and substantially parallel to the outer surface of the frame structure 312 after implantation. Having the proximal extension 331 extend alongside the outer surface of the frame structure 312 can advantageously provide torsional stability to the system (e.g., stabilize the anchor 315 relative to the frame structure 312). In some embodiments, the proximal extension 331 can be configured, for example, to terminate distal to the proximal flares of the frame structure 312 (and therefore to terminate, for example, distal to the atrium when implanted in the mitral valve annulus). In this embodiment, the proximal extension 331 can be, for example, less than 10 mm long (i.e., from the spiral 311 to the proximal-most point 335), such as less than 6 mm long, such as approximately 5 mm long.

FIG. 3C is a top-down view showing the plane 333 of the anchor 315 as it spirals around the frame structure 312.

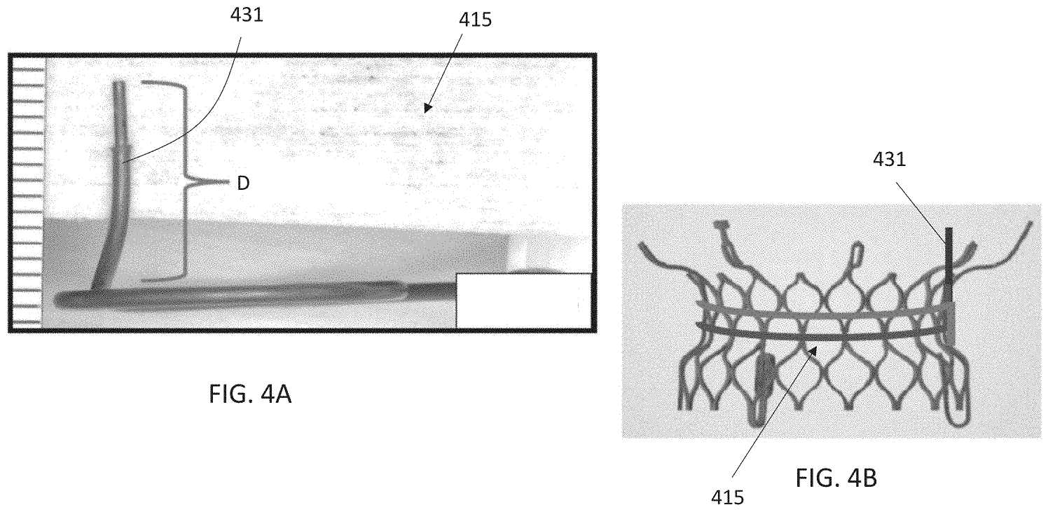

Another exemplary anchor 415 with a proximal extension 431 is shown in FIGS. 4A-4B. The extension 431 of FIGS. 4A-4B is similar to that of FIGS. 3A-3C except that the extension 431 is longer than extension 331 (e.g., has a length or extends a distance D of greater than 10 mm, such as greater than 12 mm, such as approximately 13 mm). As such, the extension 431 may extend proximally past the frame structure 412 and/or into the atrium when implanted, as shown in FIG. 4B.

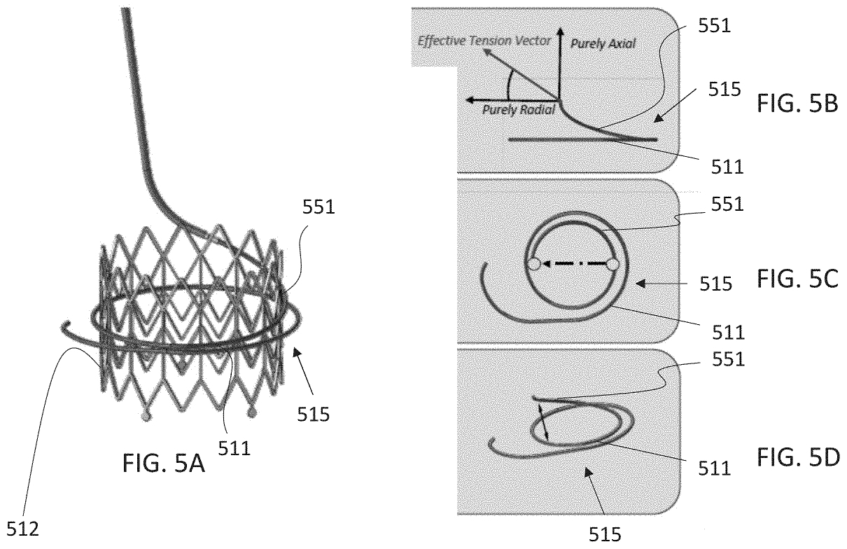

Referring to FIGS. 5A-5D, in some embodiments, the anchor 515 can include a proximal valve engagement arm 551. The valve engagement arm 551 can be similar to the proximal extension 331 of FIGS. 3A-3D except that the transition from the plane of the spiral 511 to the tip of the engagement arm 551 can be more gradual (e.g., can extend approximately 90°-270° around the circumference of the anchor 515, such as approximately 180° (see FIG. 5C). The gradual pitch or slope of the valve engagement arm 551 may advantageously improve sheathing of the anchor 515 and reduce the axial load on the delivery device. Additionally, the valve engagement arm 551 may advantageously contour closely to the valve frame 512, thereby helping to ensure coaxial alignment of the anchor 515 and the valve frame 512. Finally, the proximal end of the valve engagement arm 551 may be positioned in a substantially central point in the anchor 515 (see FIG. 5C), thereby helping to maintain the planarity of the spiral 511 relative to the anatomy is maintained even as the valve engagement arm 551 is moved axially (e.g., by the tether 78).

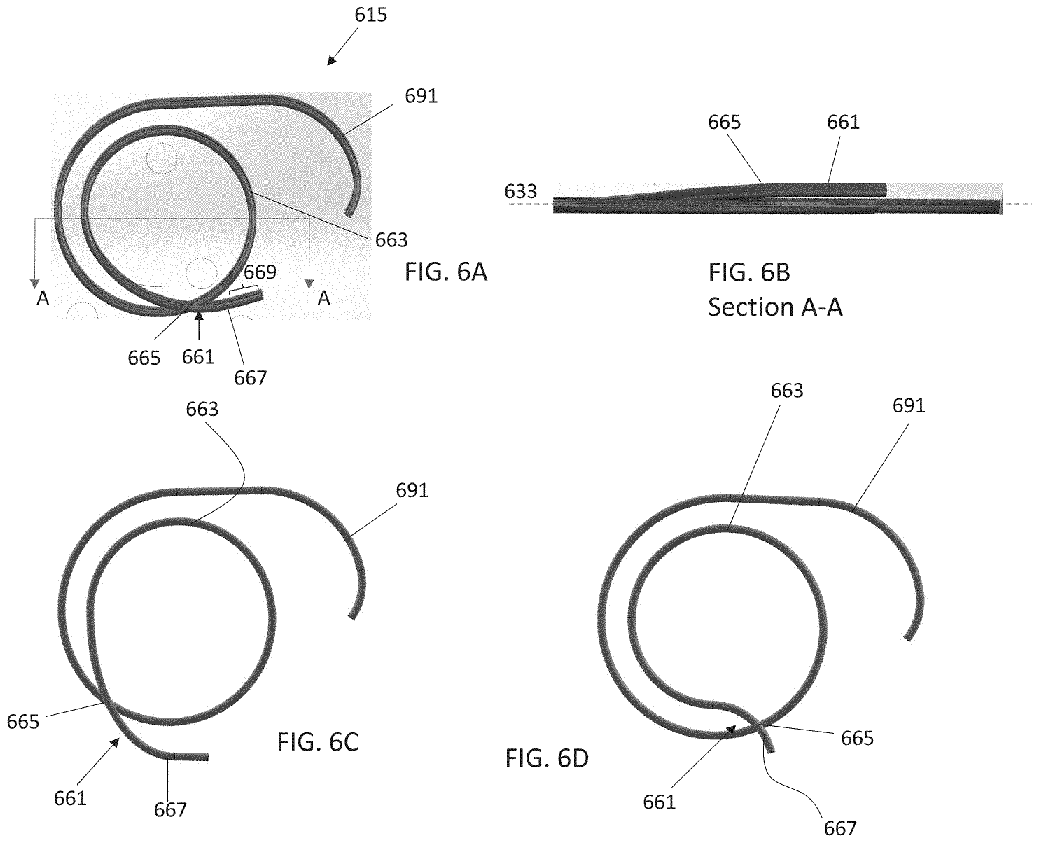

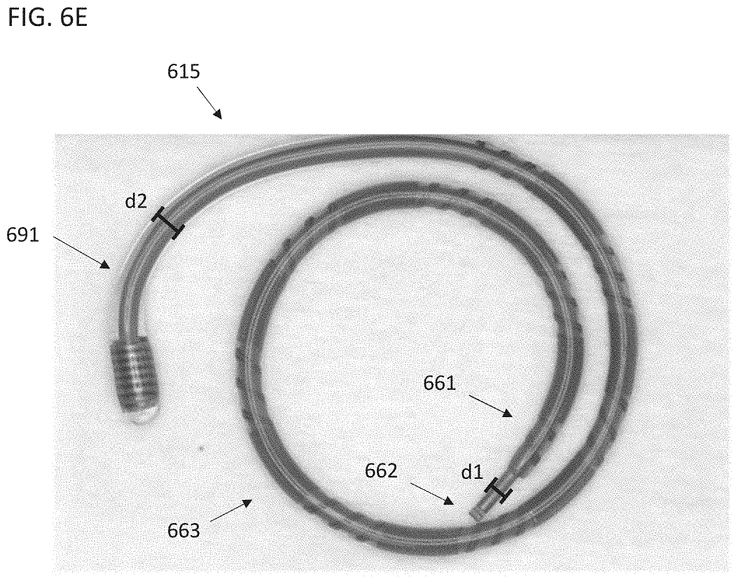

Referring to FIGS. 6A-6E, in some embodiments, the anchor 615 can include a proximal portion 661. In the embodiments of FIGS. 6A-6D, the proximal portion 661 extends radially outwards relative to the rest of the anchor 615. For example, as shown in FIGS. 6A-6D, the anchor 615 includes a distal grabber arm 691, an inner spiraled loop 663 (e.g., extending approximately 360°-540°, such as 450°), and a proximal end portion 661 that extends at a different curvature (e.g., a larger radius of curvature) than the inner spiraled loop 663. The proximal end portion 661 can extend outwards from the inner spiraled loop 663 (e.g., to a greater radial extent). In some embodiments, as shown in FIGS. 6A-6D, the proximal end portion 661 may thereby cross (e.g., cross over or overlap with) the inner spiraled loop 663 (i.e., extend beyond the perimeter or circumference of the inner spiraled loop 663) at a crossing point 665. The proximal end portion 661 can comprise one or more curvatures. The different embodiments shown in FIGS. 6A-6D are similar except that the proximal end 667 (i.e., the segment of the proximal end portion 661 that extends outside of the circumference of the inner loop 663) in the anchor 615 of FIGS. 6A-6B curves and points in the same circumferential direction as the inner loop 663, the proximal end 667 of the anchor 615 of FIG. 6C is straight and points substantially parallel to a tangent of the inner loop 663, and the proximal end 667 of the anchor 615 of FIG. 6D curves and points radially outward with respect to the circumferential direction of the inner loop 663. Configurations of the proximal end 667 such as these are expected to advantageously reduce a.) a puncture risk of the proximal end 667 into the prosthetic valve, and/or b.) any eccentricity imparted to the prosthetic valve by anchor 615.

FIG. 6B depicts a section view of the anchor 615 showing the crossing point 665 and the proximal end portion 661 positioned higher than the plane 633 defined by the inner spiraled loop 663 of the anchor 615. In some embodiments, the proximal end portion 661 is bent back down such that it is either in substantially the same plane 633 or below the plane 633.

Having the proximal end portion 661 extend and/or point radially outwards relative to the inner loop 663 can advantageously help prevent the proximal tip of the anchor 615 from puncturing through and/or getting caught in the frame structure of the prosthesis. Additionally, because the proximal end portion 661 includes a larger radius of curvature than the inner loop 663, the proximal end portion 661 can be less stiff, thereby impacting the frame structure less (e.g., putting less pressure on and/or reducing deformation of the frame structure). For example, the larger radius of curvature at the proximal end portion 661 can reduce eccentricity induced in the frame structure by the anchor 615 when the device is implanted.

In some embodiments, the proximal end portion 661 can further include a region 662 of reduced cross-sectional area (e.g., diameter, or thickness) relative to the cross-sectional diameter of the rest of the anchor 615. It should be understood that any of the embodiments described and illustrated herein can include this region having a reduced cross-sectional area. In the embodiment of FIG. 6E, the proximal end portion 661 does not extend outside of the circumference of the inner loop 663 in the anchor 615. Referring to FIG. 6E, the cross-sectional diameter d1 of the anchor at region 662 can be less than the cross-sectional diameter d2 of the anchor near the distal grabber arm 691. Reducing the cross-sectional diameter of the anchor towards the proximal end provides sections or regions of the anchor towards the proximal end that are more flexible (or less stiff) than the other regions of the anchor with a larger cross-sectional diameter. In some embodiments, as shown, the region 662 of reduced cross-sectional diameter can be a sudden or abrupt change from the larger cross-sectional diameter of the rest of the anchor. In other embodiments, the anchor can gradually reduce in cross-sectional diameter as the anchor extends from the distal end to the proximal end. For example, the anchor can have its greatest cross-sectional diameter at the distal end (near the distal grabber arm), gradually reducing in cross-sectional diameter along the length of the anchor until the proximal end of the anchor. In some embodiments, the region of reduced cross-sectional diameter comprises less than half a turn (e.g., 180 degrees) of the anchor. In other embodiments, the region of reduced cross-sectional diameter comprises less than a quarter of a turn (e.g., 90 degrees) of the anchor. As described above, the transition from larger cross-sectional diameter to smaller cross-sectional diameter can be abrupt or can be gradual. The changing cross-section or diameter of the anchor can help with delivery of the anchor and change the load that the anchor applies to the anatomy or frame structure at different portions of the anchor.

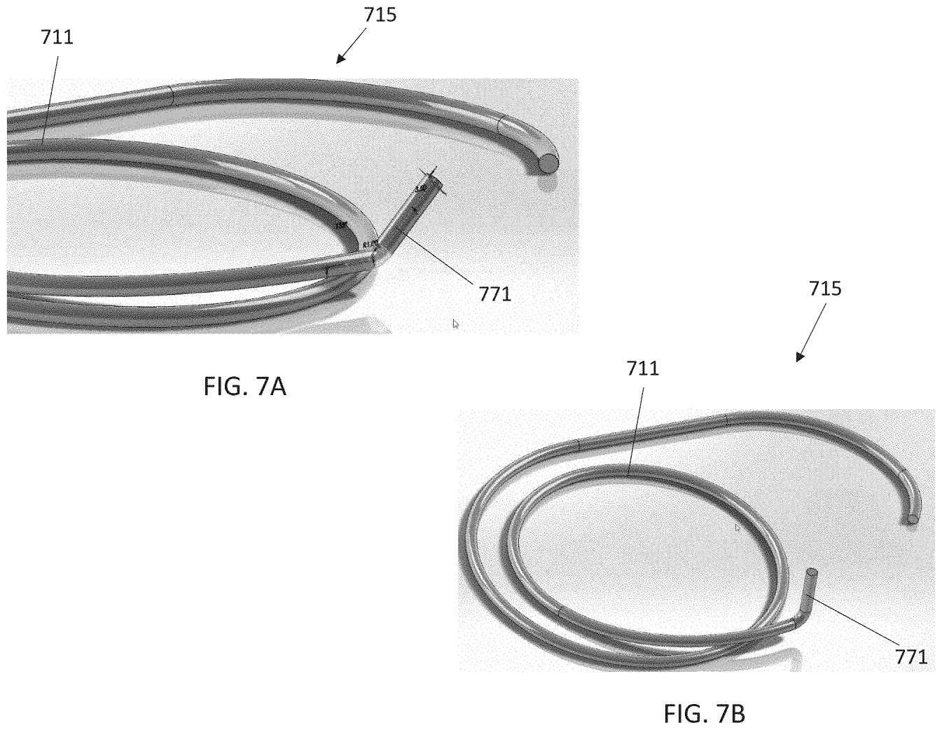

Referring to FIGS. 7A-7B, in some embodiments, the anchors 715 described herein can include a combined proximal extension that extends out of plane and a proximal end portion that extends radially outwards with respect to the circumferential direction of the inner loop of the spiral. The embodiments of the anchor 715 of FIGS. 7A-7B are similar except that the proximal end 771 of anchor 715 of FIG. 7A extends at a substantially 60°-80° angle with respect to the plane of the spiral 711 while the proximal end 771 of FIG. 7B extends at a substantially 85°-95° angle with respect to the plane of the spiral 711. The tilt or angle of the proximal end 771 as shown in these embodiments further avoids puncture risk on the valve and reduces eccentricity imparted to the valve during delivery/manipulation. As described above, the proximal arm or proximal end can further provide the benefit of manipulating the axial height of the anchor relative to the frame structure during delivery, while reducing rotational forces imparted to the anchor.

Any of the embodiments described herein may include short and straight engagement section at the proximal-most end thereof configured to enable simple engagement with a release mechanism (e.g., with a release mechanism from the tether 78). For example, the anchor 615 of FIG. 6A shows a straight section 669 at the proximal end thereof. The straight section 669 can be less than 5 mm, such as less than 4 mm, such as approximately 3.5 mm in length.

In some embodiments, the proximal extension, proximal engagement arm, and/or proximal end portions described herein can extend and/or point distally rather than proximally (e.g., to interact with a delivery device positioned within the ventricle).

Additional elements of valve prostheses and anchors are described in PCT Application No. PCT/US2019/047542 filed on Aug. 21, 2019, PCT Application No. PCT/US2019/057082 filed on Mar. 19, 2019, PCT Application No. PCT/US2019/068088 filed on Dec. 20, 2019, and PCT Application No. PCT/US2020/23671, the entireties of which are incorporated by reference herein in their entireties.

It should be understood that any feature described herein with respect to one embodiment can be substituted for or combined with any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A prosthesis for treating a diseased native valve, the prosthesis comprising:
a frame structure having a plurality of leaflets therein; and
a spiral anchor configured to extend around an outer circumference of the frame structure, the spiral anchor comprising a spiral portion and a proximal portion that extends radially outwards relative to a circumference of the spiral portion.

2. The prosthesis of claim 1, wherein the proximal portion crosses the spiral portion to extend beyond the circumference of the spiral portion.

3. The prosthesis of claim 1, wherein the proximal portion further extends at a 60°-90° angle relative to a plane of the spiral portion.

4. The prosthesis of claim 1, wherein the proximal portion has a length of up to 5 mm.

5. The prosthesis of claim 1, wherein the proximal portion has a length of up to 10 mm.

6. The prosthesis of claim 1, wherein the proximal portion has a smaller cross-sectional area than that of the spiral portion.

7. The prosthesis of claim 6, wherein the proximal portion comprises an abutment at the transition between the proximal portion and the spiral portion.

8. The prosthesis of claim 1, further comprising a delivery tether configured to releasably engage with the proximal portion of the spiral anchor.

9. The prosthesis of claim 1, wherein the spiral portion is substantially contained within the plane.

10. A prosthesis for treating a diseased native valve, the prosthesis comprising:
a frame structure having a plurality of leaflets therein; and
a spiral anchor configured to extend around an outer circumference of the frame structure, the spiral anchor comprising a spiral portion and a proximal extension at a proximal end of the spiral portion, the proximal extension extending at a 60°-90° angle relative to a plane of the spiral portion.

11. The prosthesis of claim 10, wherein the proximal extension is configured to extend along an outer surface of the frame structure.

12. The prosthesis of claim 10, wherein the frame structure comprises a flared proximal portion, the proximal extension terminating distal to the flared proximal portion.

13. The prosthesis of claim 10, wherein the proximal extension is configured to position the proximal end to extend at a 60°-90° angle relative to the plane of the spiral portion.

14. The prosthesis of claim 10, wherein the proximal extension is less than 10 mm long.

15. A prosthesis for treating a diseased native valve, the prosthesis comprising:
a frame structure having a plurality of leaflets therein; and
a spiral anchor configured to extend around an outer circumference of the frame structure, the spiral anchor having a proximal portion that is more flexible than a distal portion.

16. The prosthesis of claim 15, wherein the proximal portion is thinner or has a smaller cross-sectional area than that of the distal portion.

17. The prosthesis of claim 15, wherein a cross-sectional area of the spiral anchor increases along its length from the proximal portion to the distal portion.

18. The prosthesis of claim 17, wherein the increasing cross-sectional area of the spiral portion is gradual along an angular extent of the spiral portion.

19. The prosthesis of claim 17, wherein the increasing cross-sectional area of the spiral portion is gradual along a linear extent of the spiral portion.

20. The prosthesis of claim 17, wherein the increasing cross-sectional area of the spiral portion is abrupt along an angular extent of the spiral portion.

21. The prosthesis of claim 17, wherein the increasing cross-sectional area of the spiral portion is abrupt along a linear extent of the spiral portion.

22. A method of delivering a valve prosthesis, comprising the steps of:
coupling a delivery tether to a proximal portion of a spiral anchor;
providing the spiral anchor into a delivery sheath;
advancing the delivery sheath to a target tissue location;
deploying the spiral anchor from the delivery sheath into a plane to encircle one or more mitral leaflets and chordae at the target location;
delivering a frame structure over the delivery tether into place within the spiral anchor; and
adjusting an axial position of the spiral anchor relative to the frame structure by the delivery tether while maintaining the planarity of the spiral anchor.

23. The method of claim 22, wherein the axial position of the spiral anchor is adjusted without rotating the spiral anchor relative to the frame structure.

24. The method of claim 22, wherein the axial position of the spiral anchor is adjusted without increasing a radius of curvature of the spiral anchor.

25. The method of claim 22, wherein the axial position of the spiral anchor is adjusted without increasing a radius of curvature of a proximal end portion of the spiral anchor.
